(19) United States
(12) Patent Application Publication
(10) Pub. No.: US 2023/0372742 A1
(43) Pub. Date: Nov. 23, 2023

(54) ACOUSTIC SHOCK WAVE TREATMENT FOR ERECTILE DYSFUNCTION

(71) Applicant: SoftWave Tissue Regeneration Technologies, LLC, Kennesaw, GA (US)

(72) Inventors: John F. Warlick, Woodstock, GA (US); Irwin Goldstein, San Diego, CA (US)

(21) Appl. No.: 18/196,636

(22) Filed: May 12, 2023

Related U.S. Application Data
(60) Provisional application No. 63/344,720, filed on May 23, 2022.

(51) Int. Cl. A61N 7/00

(52) U.S. Cl. CPC A61N 7/00

(57) ABSTRACT

A method of treatment for a penis of an adult postpubertal male patient, the treatment has of the steps of: causing an erection of a penis; activating an acoustic shock wave generator or source to emit low energy or unfocused acoustic shock waves and subjecting the erect penis to the acoustic shock waves stimulating said erect penis, the erect penis is positioned within a path of the emitted shock waves stimulating a cellular response.

### FIELD OF THE INVENTION

The present invention relates to improved treatments of appendages for repairing or regenerating reproductive cells, tissues or vessels to overcome various degenerative conditions.

### BACKGROUND OF THE INVENTION

Treating conditions of male infertility and impotency is extremely important for those affected by conditions such as Erectile Dysfunction.

In some cases, men take drugs to enhance their sexual performance and stamina. These medications, while beneficial, have some adverse consequences. The use of testosterone enhancing steroids for example can lead to serious complications.

In U.S. Pat. No. 7,601,127 B2, the inventor found that treating male genitalia including the penis with acoustic shock waves could overcome such conditions such as impotency, including erectile dysfunction.

The present invention has found a way to more effectively treat these conditions with faster and more reliable results by applying acoustic shock waves directly to the penis as discussed hereinafter.

Erectile Dysfunction (ED) is an ailment in which a male is unable to achieve or sustain an erection suitable for sexual intercourse. A number of factors are believed to play a role in or be directly responsible for ED, including obesity, blood pressure, chronic illnesses such as diabetes, poor blood flow to the penis, smoking tobacco, alcoholism, and side-effects of other medications.

Treatments for ED currently include cessation of potential causes such as smoking tobacco and consumption of alcohol, hormone (testosterone) replacement, surgery, and administration of pharmaceuticals such as vardenafil, tadalafil, and sildenafil. Some of these pharmaceuticals are controversial for their incompatibility with nitrate drugs, and for their unwanted side-effects, such as effects on vision (blurring, loss of vision) and priapism.

One objective of the present invention is to assist in regeneration of the male reproductive tissues and organs to correct at least partially degenerative conditions resuiting from aging or disease. Another objective of the present invention is to stimulate the healing process of the male reproductive system after corrective surgery in cases where surgery is required to repair a defect in reproductive tissue or organs. Another objective is to stimulate tissue revascularization and blood flow effectively to improve either performance or sensitivity to sexual contact thereby enhancing sexual experience for a male. These and other objectives are achieved using the inventive technology described herein.

### SUMMARY OF THE INVENTION

A method of treatment for a penis of an adult postpubertal male patient, the treatment has of the steps of: causing an erection of a penis; activating an acoustic shock wave generator or source to emit low energy or unfocused acoustic shock waves, wherein the acoustic shock waves are waves having amplitudes above 0.1 MPa and rise times of the amplitude are below 100 nano-seconds with a duration of a shock wave being below 3 micro-seconds for the positive part of a cycle and wherein the pressure pulses are an acoustic pulse which includes several cycles of positive and negative pressure with amplitudes of the positive part of such a cycle being above 0.1 MPa and the pressure pulse time duration is from below a microsecond to about a second, rise times of the positive part of the first pressure cycle is in the range of nano-seconds up to several milli-seconds; and subjecting the erect penis to the acoustic shock waves stimulating said erect penis, the erect penis is positioned within a path of the emitted shock waves stimulating a cellular response.

The method of treatment wherein the emitted shock waves or pressure pulses can be convergent, divergent, planar or near planar. The method of treatment wherein the emitted shock waves or pressure pulses can be convergent having one or more geometric focal volumes or points located at a distance X, X being defined as the distance from an exit window to the one or more focal volumes or points from the generator or source, the erect penis being positioned at the distance X or less than the distance X from the exit window source.

A second embodiment method of treatment for increasing testosterone levels and increasing sperm count and viability in an adult postpubertal male patient has of the steps of: testing the testosterone and sperm count and viability of a patient prior to treatment to establish a pre-treatment initial baseline level; activating an acoustic shock wave or pressure pulse generator or source to emit low energy or unfocused acoustic shock waves or pressure pulses, wherein the acoustic shock waves are waves having amplitudes above 0.1 MPa and rise times of the amplitude are below 100 nano-seconds with a duration of a shock wave being below 3 micro-seconds for the positive part of a cycle and wherein the pressure pulses are an acoustic pulse which includes several cycles of positive and negative pressure with amplitudes of the positive part of such a cycle being above 0.1 MPa and the pressure pulse time duration is from below a microsecond to about a second, rise times of the positive part of the first pressure cycle is in the range of nano-seconds up to several milli-seconds; subjecting a testicle through the scrotum to the acoustic shock waves or pressure pulses stimulating said testicle to increase testosterone levels and increase sperm count and viability, wherein the testicle is positioned within a path of the emitted shock waves stimulating a cellular response without creating cavitation bubbles insuring no hemorrhaging; testing the testosterone level and sperm count and viability of the patient with-in 72 hours after subjecting the testicle to the acoustic shock waves or pressure pulses to establish a post treatment level; and comparing the pre-treatment baseline level to the post treatment level tested after treatment to establish any increase.

### Definitions

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Erectile dysfunction (ED)” is an ailment in which a male is unable to achieve or sustain an erection suitable for sexual intercourse.

“extracorporeal” occurring or based outside the living body.

A “generalized paraboloid” according to the present invention is also a three-dimensional bowl. In two dimensions (in Cartesian coordinates, x and y) the formula yⁿ=2px [with n being≠2, but being greater than about 1.2 and smaller than 2, or greater than 2 but smaller than about 2.8]. In a generalized paraboloid, the characteristics of the wave fronts created by electrodes located within the generalized paraboloid may be corrected by the selection of (p (−z,+z)), with z being a measure for the burn down of an electrode, and n, so that phenomena including, but not limited to, burn down of the tip of an electrode (−z,+z) and/or disturbances caused by diffraction at the aperture of the paraboloid are compensated for.

“impotence” an abnormal physical or psychological state of a male characterized by inability to copulate because of failure to have or maintain an erectioncalled also erectile dysfunction.

A “paraboloid” according to the present invention is a three-dimensional reflecting bowl. In two dimensions (in Cartesian coordinates, x and y) the formula y²=2px, wherein p/2 is the distance of the focal point of the paraboloid from its apex, defines the paraboloid. Rotation of the two-dimensional figure defined by this formula around its longitudinal axis generates a de facto paraboloid.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude can be below 1000 ns, preferably at or below 100 ns. The duration of a shock wave is typically below 1-3 micro-seconds (μs) for the positive part of a cycle and typically above some micro-seconds for the negative part of a cycle.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Washington 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

Wave energy or energy flux density: the measurement of energy flux density is defined as the energy directed toward the target or region being treated. This is not energy at the gap between electrodes, but rather the energy transmitted toward the patient's tissue through the skin. Important to distinguish that the energy levels discussed pertain to the energy delivered to the targeted tissues and not at the discharge point between the electrode tips. Spherical waves have a huge amount of energy produced between the tips to deliver adequate energy to the targeted tissues since they do not have the advantage of a lens.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The invention will be described by way of example and with reference to the accompanying drawings in which:

FIG. 1 is a simplified depiction of a pressure pulse/shock wave (PP/SW) generator with focusing wave characteristics.

FIG. 2 is a simplified depiction of a pressure pulse/shock wave generator with plane wave characteristics.

FIG. 3 is a simplified depiction of a pressure pulse/shock wave generator with divergent wave characteristics.

FIG. 4 is a perspective view of a shock wave generator device.

FIG. 5 is a view of the erect penis with an acoustic shock wave applicator shown treating the erect penis with acoustic shock waves with a contrasting view in dashed lines of a non-erect penis.

FIG. 6 is a graph showing an exemplary ultrasound wave pattern.

FIG. 7 is a graph of an exemplary acoustic shock wave pattern.

### DETAILED DESCRIPTION OF THE INVENTION

The present invention treats a male penis that is exhibiting an erection. The treatment involves directing acoustic shock waves or pressure pulses through the erect penis to eliminate or reduce ED symptoms.

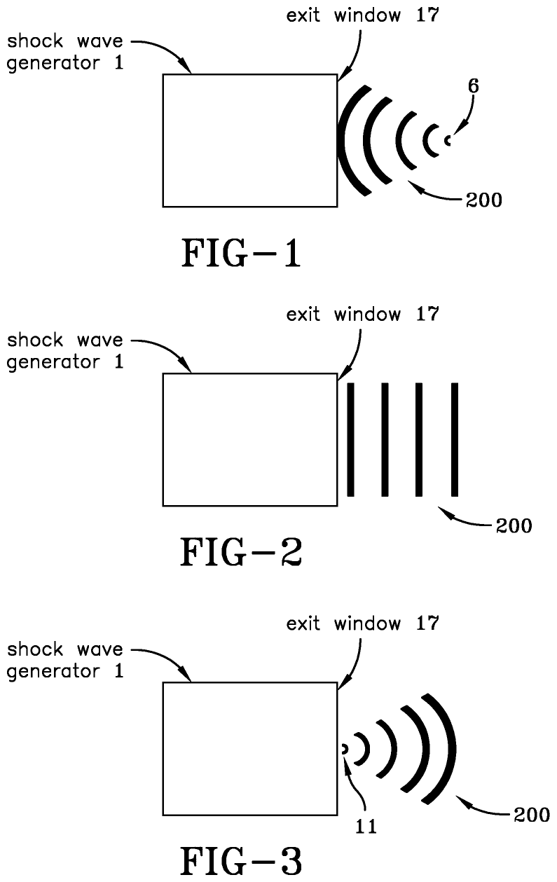

With reference to the figures, FIGS. 1-3 show various shock waves 200 being transmitted from a shock wave generator 1. FIG. 1 is a focused shock wave wherein the shock wave pattern emits from an exit window 17 and focuses to a point or localized area 6 as illustrated. The wave form 200 is considered a converging wave form. In FIG. 2, the shock wave emits from the exit window 17 in a planar fashion wherein the wave forms 200 are transmitted parallel to the exit window 17 and transmitted into the patient. In FIG. 3, the wave form 200 is emitted from the exit window 17 in such a fashion that the focal point is near the exit window 17 and the wave form 200 expands outwardly, this is considered a divergent wave form wherein the wave form expands as it leaves the exit window 17 in a diverging pattern.

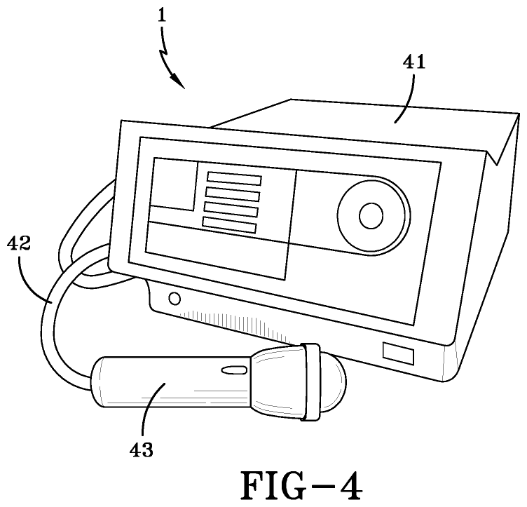

With reference to FIG. 4, an exemplary acoustic shock wave apparatus1 is illustrated. The shock wave apparatus 1 has a generator 41 connected by a hose with a hose extending from the shock wave generator 41 to an applicator 43. The applicator 43 as illustrated has a body that enables a technician to hold the applicator 43 and as illustrated this applicator is an electrohydraulic that is filled with fluid to facilitate the transmission of the shock waves. The fluid expands a flexible membrane in such a fashion that the membrane extends outwardly in a balloon shape fashion as illustrated in FIG. 4.

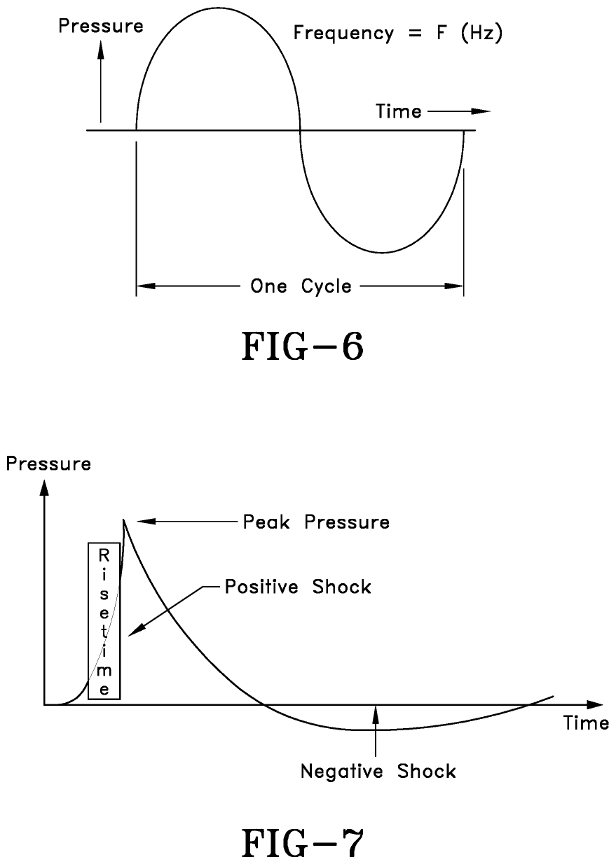

An important aspect of the present invention is that the pressure waves from the acoustic shock wave generator have an asymmetric type wave form with a very high peak pressure that occurs over a very short rise time. The positive shock is transmitted in a very quick fashion as defined which defines the features of an acoustic shock wave or pressure pulse. The negative portion of the wave is longer in duration and encompasses the rest of the wave form as shown in FIG. 7. This is unlike ultrasound wave forms which are symmetrical, sinusoidal in shape as illustrated in FIG. 6. The main difference between an acoustic shock wave and an ultrasound wave is that there is no heat generated in the asymmetric type acoustic shock wave whereas there is heat generation in the ultrasound wave. The ultrasound wave therefore is considered inferior for the purposes of erectile dysfunction or any other treatment for that matter compared to the use if the electrohydraulic acoustic shock waves emitted from the applicator 43. The applicator 43 as shown is electrohydraulic, but it could be ballistic, piezoelectric or any other form of applicator.

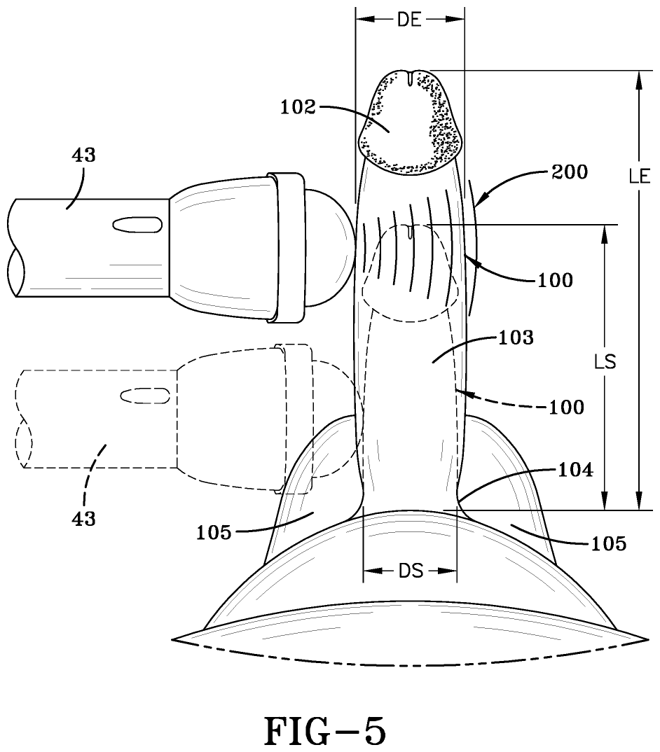

An important aspect of the present invention is that, as illustrated in FIG. 5, the shock wave applicator 43 when applied to a penis 100 for treating erectile dysfunction is generally held in one hand by the technician and the other hand extends an index finger along the shaft of the penis 100. It has been determined that during these treatments, the acoustic shock waves 200 on a flaccid penis 100, that is a non-erect penis, is such that the wave transmission extends beyond the diameter DS of the non-erect penis 100. In fact, the non-erect penis 100, shown in dashed lines for comparison, has a diameter DS of approximately greater than 2 cm typically. Whereas the acoustic shock wave applicator 43 membrane and exit window that emits most of the energy is approximately 5 cm diameter. When this occurs, the technician who supports the shaft 103 of the penis 100 has an index finger lying along the shaft 103 to support the penis 100 and as the shock wave transmission is transmitted through the flaccid penis 100 the energy gets transmitted to the technician and the technician is actually treating his hand with shock waves due to the fact that the non-erect penis is sufficiently small in diameter that it allows for energy to be transmitted past and through the soft penis 100. After having been treating penises of this type, it was noticed that if a penis was made to have an erection, the shaft 103 of the penis increases by approximately one third to greater than 3 cm, typically about 5 cm which is approximately the size of the applicator head for the emitted energy pulse being transmitted. In this case, the technician is holding the applicator 43 and the index finger (not shown) is extending along the shaft of the penis, the energy transmission is primarily absorbed through the erect penis 100 due to the increased size/diameter DE.

This erection of the penis 100 not only increases the diameter DE which approximates the size of the wave form 200 being transmitted but also increases the length from LS for the non-erect penis to LE for the erect penis length. This enables the device to be able to transmit as one can appreciate when the membrane of the applicator 43 is pressed against the erect penis 100, the entire membrane can be moved along the shaft 103 from the head 102 to the base 104 near the testicles 105 to treat the penis 100 for erectile dysfunction. As the technician rotates the applicator 43 about the shaft 103 of the penis 100, it can be moved diametrically around the shaft of the penis as well as along the length LE of the penis 100 ensuring the entire length of the penis is being effectively treated with acoustic shock waves.

These acoustic shock waves create increased vascularization and blood flow. Since the patients being treated are afflicted with erectile dysfunction, it is very difficult to maintain an erection. Accordingly, the patient could be given a simple treatment of taking a medication that will assist in maintaining an erection for a period of time or preferably is given an injection with a chemical that will artificially create the erection.

A chemical or artificial erection involves injection of medications such as alprostadil, a formulation of prostaglandin E, available under various trade names. For example, one medication is a mixture of prostaglandin, phentolamine, and papaverine into the base of the penis causing an erection.

Unfocused LiSWT reflected by parabolic applicator, Creates linear beam of unfocused energy, Compression wave travels through tissue, Amplitude+intensity decreases with distance attenuation due to: Acoustic energy conversion to other form−heat/cavitation (absorption), Redirection of energy via scatter/reflection/refraction/diffraction/divergence, Energy absorbed by penile tissues ->heat and cavitation, Microtrauma leads to neovascularization, tissue regeneration, reduction of inflammation stem cell activation, At tissue interface energy reflection vs transmission depends on difference in acoustic impedance between tissues (impedance matching), Small impedance mismatch=most energy transmitted little reflected/refracted/scattered, Large impedance mismatch=most energy reflected/refracted/scattered−little transmitted. Very small reflectors like RBCs (dimensions<<ultrasonic wavelength) also produce scattering—‘Rayleigh scatterers”.

Hypothesis: erect penile tissue energy absorption >>flaccid penile tissue absorption due to: Increased impedance mismatch at blood-tissue interface of erect penis, Increased scatter/refraction/diffraction due to more RBC/Rayleigh scatterers, Larger erect penis diameter brings focal into tissue rather than outside penis, Acoustic wave propagates inside erect tissue instead of quickly through flaccid tissue. See the lacunar spaces increase in size during erection, These increased size spaces increases acoustic impedance mis-matching

In the extracorporeal shock wave or pressure pulse method of treating an organ or tissue, the administered shock waves or pressure pulses are directed to a treatment location or target site on the organ or tissue. As used herein, “near” recognizes that the emitted shock waves or pressure pulses are transmitted through the organ or tissue, preferably at or in close proximity to the treatment location or site.

Assuming the target area is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent or near planar and having a low-pressure amplitude and density in the range of 0.00001 mJ/mm² to 1.0 mJ/mm² or less, most typically below 0.2 mJ/mm². These are typically generated by spherical or radial wave generators, ballistic or electrohydraulic wave or piezoelectric shock wave generators. The focused source can use a focused beam of waves or can optionally use a diffusing lens or have a far-sight focus to minimize if not eliminate having the localized focus zone within the organ or tissue. Preferably the focused shock waves are used at a similarly effective low energy transmission or alternatively can be at higher energy but wherein the organ or tissue target site is disposed pre-convergence inward of the geometric focal point of the emitted wave transmission. Understanding the higher the energy used, the more sensation of bruising the organ or tissue. In these cases, cavitation can and often does occur as well as bruising and come cell damage. This is preferably and easily avoidable.

These shock wave energy transmissions are effective in stimulating a cellular response and in some cases, such as unfocused low energy, and even low energy focused emissions can be accomplished without creating the localized hemorrhaging caused by rupturing cavitation bubbles in the organ or tissue of the target site. This effectively ensures the harvested organ or tissue does not have to experience the sensation of cellular damage so common in the higher energy focused wave forms having a focal point at or within the targeted treatment site. Higher energy acoustic shock waves or pressure pulses including focused waves can be used, but with care to avoid such damage.

The target site may be such that the organ or tissue or the generating source must be reoriented relative to the site and a second, third or more treatment dosage can be administered. At a low energy, the common problem of localized hemorrhaging is reduced making it more practical to administer multiple dosages of waves from various orientations to further optimize the treatment and cellular stimulation of the target site. Alternatively, focused high energy multiple treatments can be equally effective, but with some risk to organ or tissue bruising. The use of low energy focused or unfocused waves at the target site enables multiple sequential treatments. Alternatively, the wave source generators may be deployed in an array wherein the subject organ or tissue is effectively enveloped or surrounded by a plurality of low energy wave source generators which can be simultaneously bombarding the target site from multiple directions. Such arrays include linear type devices.

The goal in such treatments is to provide 100 to 3000 acoustic shock waves or pressure pulses. Typically, at a voltage of 14 kV to 28 kV across a spark gap generator in a single treatment preferably or one or more adjuvant treatments by targeting the site directly by impinging the emitted waves toward the male penis.

The present method does not rely on precise site location per se. The physician's general understanding of the anatomy of the penile organ or tissue should be sufficient to locate a desirable direct path or to the target site to attack the condition being treated. The treated area can withstand a far greater number of shock waves based on the selected energy level being emitted. For example, at very low energy levels the stimulation exposure can be provided over prolonged periods as much as 20 minutes if so desired. At higher energy levels the treatment duration can be shortened to less than a minute, less than a second if so desired. The selected treatment dosage can include the avoidance or minimization of cell hemorrhaging and other kinds of damage to the cells or tissue while still providing a stimulating cellular release activation of upregulation of the antimicrobial peptide LL37, a protein that can bind with RNA to destroy any infections, and also VEGF and other growth factors and can also be used to modulate and regulate spermal secretions from the specific targeted gland or penis by emitting waves to a desired direct path.

The underlying principle of these sound wave therapy methods is to stimulate the penile organ or tissue. This is accomplished by deploying shock waves to stimulate strong cells in the tissue to activate a variety of responses, more particularly those that reduce inflammation and stop any infections. The sound waves including acoustic shock waves or pressure pulses transmit or trigger what appears to be a cellular communication throughout the entire anatomical structure of the penile organ or tissue, this activates a generalized cellular response at the treatment or target site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced but also the number of applied shock wave impulses can be lowered from several thousand to as few as one or more pulses and still yield a beneficial stimulating response if desired.

The biological model motivated the design of sources with low pressure amplitudes and energy densities. First: spherical waves generated between two tips of an electrode; and second: nearly even waves generated by generated by generalized parabolic reflectors. Third: divergent shock front characteristics are generated by an ellipsoid. Unfocused sources are preferably designed for extended two dimensional areas/volumes like skin. The unfocused sources can provide a divergent wave pattern or a nearly planar wave pattern and can be used in isolation or in combination with focused wave patterns yielding to an improved therapeutic treatment capability that is non-invasive with few if any disadvantageous contraindications. Alternatively, a focused wave emitting treatment may be used wherein the focal point extends to the target site. In any event, the beam of acoustic waves transmitted needs to project in a large enough zone or area to stimulate or modulate the cells in the erect penis.

In one embodiment, the method of treatment has the steps of, generating either focused shock waves or unfocused shock waves, of directing these shock waves to the treatment site; and applying a sufficient number of these shock waves to induce activation of one or more growth factor or anti-microbial peptides like LL37, thereby inducing or accelerating a modulated adjustment to induce the host cells of the penis to attack any infection or disease. In this case, preferably to overcome ED.

The shock waves can be of a low peak pressure amplitude and density. Typically, the energy density values range as low as 0.000001 mJ/mm² and having a high end energy density of below 1.0 mJ/mm², preferably 0.40 mJ/mm² or less, more preferably 0.20 mJ/mm² or less. The peak pressure amplitude of the positive part of the cycle should be above 1.0 and its duration is below 1-3 microseconds.

The treatment depth can vary from the surface to the full depth of the penile organ or tissue and the treatment site can be defined by a much larger treatment area. The above methodology is particularly well suited for surface as well as sub-surface soft tissue treatments in penile organs or tissue.

An exemplary treatment protocol could have emitted shock waves in a broad range of 0.01 mJ/mm² to 3.0 mJ/mm² and 200-2500 pulses per treatment with a treatment schedule of 1-3 treatments being repeated over several weeks.

In the shock wave method of treating a tissue, an organ or the entire body of a patient diagnosed with infertility or impotence requires the patient to be positioned in a convenient orientation to permit the source of the emitted waves to most directly send the waves to the target site to initiate shock wave stimulation of the target area with minimal, preferably no obstructing features in the path of the emitting source or lens. Assuming the target area is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent, planar or near planar and having a low pressure amplitude and density in the range of 0.00001 mJ/mm<2> to 1.0 mJ/mm<2> or less, most typically below 0.2 mJ/mm<2>. The focused source preferably can use a diffusing lens or have a far-sight focus to minimize if not eliminate having the localized focus point within the tissue. Preferably the focused shock waves are used at a similarly effective low energy transmission or alternatively can be at higher energy but wherein the tissue target site is disposed pre-convergence inward of the geometric focal point of the emitted wave transmission.

These shock wave energy transmissions are effective in stimulating a cellular response and can be accomplished without creating the cavitation bubbles in the tissue of the target site. This effectively insures the penile tissue or organ does not have to experience the sensation of hemorrhaging so common in the higher energy focused wave forms having a focal point at or within the targeted treatment site.

Due to the wide range of beneficial treatments available it is believed preferable that the optimal use of one or more wave generators or sources should be selected on the basis of the specific application. Wherein relatively small target sites may involve a single wave generator placed on an adjustable manipulator arm. A key advantage of the present inventive methodology is that it is complimentary to conventional medical procedures. In the case of any operative surgical procedure the surgical area of the patient can be bombarded with these low energy waves to stimulate cellular release of healing agents and growth factors. This will dramatically reduce the healing process time. Most preferably such patients may be provided more than one such treatment with an intervening dwell time for cellular relaxation prior to secondary and tertiary post operative treatments.

The underlying principle of these shock wave therapy methods is to stimulate the body's own natural healing capability. This is accomplished by deploying shock waves to stimulate strong cells in the penile tissue to activate a variety of responses. The acoustic shock waves transmit or trigger what appears to be a cellular communication throughout the entire anatomical structure, this activates a generalized cellular response at the treatment site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced but also the number of applied shock wave impulses can be lowered from several thousand to as few as one or more pulses and still yield a beneficial stimulating response.

The use of shock waves as described above appears to involve factors such as thermal heating, light emission, electromagnetic field exposure, chemical releases in the cells as well as a microbiological response within the cells. Which combination of these factors plays a role in stimulating healing is not yet resolved. However, there appears to be a commonality in the fact that growth factors are released which applicants find indicative that otherwise dormant cells within the tissue appear to be activated which leads to the remarkable ability of the targeted penile organ or tissue to generate new growth or to regenerate weakened vascular networks in for example the reproductive system. This finding leads to a complimentary use of shock wave therapy in combination with stem cell therapies that effectively activate or trigger stem cells to more rapidly replicate enhancing the ability to harvest and culture more viable cells from the placenta, a nutrient culture of said stem cells, or other sources. The ability to stimulate stem cells can occur within the patient's own body activating the naturally occurring stem cells or stem cells that have been introduced to the patient as part of a treatment beneficially utilizing stem cells. This is a significant clinical value in its own right and is critical in attempts to overcome conditions of infertility.

The use of shock wave therapy requires a fundamental understanding of focused and unfocused shock waves, coupled with a more accurate biological or molecular model.

Focused shock waves are focused using ellipsoidal reflectors in electromechanical sources from a cylindrical surface or by the use of concave or convex lenses. Piezoelectric sources often use spherical surfaces to emit acoustic pressure waves which are self focused and have also been used in spherical electromagnetic devices.

The biological model proposed by Wolfgang Schaden provides a whole array of clinically significant uses of shock wave therapy.

Accepting the biological model as promoted by W. Schaden, the peak pressure and the energy density of the shock waves can be lowered dramatically. Activation of the body's healing mechanisms will be seen by in growth of new blood vessels and the release of growth factors.

The biological model motivated the design of sources with low pressure amplitudes and energy densities. First: spherical waves generated between two tips of an electrode; and second: nearly even waves generated by generalized parabolic reflectors. Third: divergent shock front characteristics are generated by an ellipsoid behind F2. Unfocused sources are preferably designed for extended two dimensional areas/volumes like skin. The unfocused sources can provide a divergent wave pattern a planar or a nearly planar wave pattern and can be used in isolation or in combination with focused wave patterns yielding to an improved therapeutic treatment capability that is non-invasive with few if any disadvantageous contraindications. Alternatively a focused wave emitting treatment may be used wherein the focal point extends preferably beyond the target treatment site, potentially external to the patient. This results in the reduction of or elimination of a localized intensity zone with associated noticeable pain effect while providing a wide or enlarged treatment volume at a variety of depths more closely associated with high energy focused wave treatment. The utilization of a diffuser type lens or a shifted far-sighted focal point for the ellipsoidal reflector enables the spreading of the wave energy to effectively create a convergent but off target focal point. This insures less tissue trauma while insuring cellular stimulation to enhance the healing process and to effectively remodel the reproductive tissues or organs of the patient.

This method of treatment has the steps of, locating a treatment site, generating either convergent diffused or far-sighted focused shock waves or unfocused shock waves, of directing these shock waves to the treatment site; and applying a sufficient number of these shock waves to induce activation of one or more growth factors thereby inducing or accelerating healing and tissue and organ remodeling or repair.

The unfocused shock waves can be of a divergent wave pattern, planar or near planar pattern preferably of a low peak pressure amplitude and density. Typically, the energy density values range as low as 0.000001 mJ/mm² and having a high end energy density of below 1.0 mJ/mm², preferably 0.20 mJ/mm² or less. The peak pressure amplitude of the positive part of the cycle should be above 1.0 and its duration is below 1-3 microseconds.

The treatment depth can vary from the surface to the full depth of the treated organ. The treatment site can be defined by a much larger treatment area than the 0.10-3.0 cm² commonly produced by focused waves. The above methodology is particularly well suited for surface as well as sub-surface soft tissue organ treatments as is found in the regions of the male reproductive system.

The above methodology is valuable in generation of tissue, vascularization and may be used in combination with stem cell therapies as well as regeneration of tissue and vascularization. While the above listed indications cited above are not exhaustive nor intended to be limiting, it is exemplary of the wide range of beneficial uses of low energy and amplitude unfocused divergent, planar or nearly planar shock waves, convergent shock waves, diffused shock waves or a combination of shock wave types in the treatment of humans and other mammals that are infertile or impotent.

A most significant method of preventive medicine can be practiced that is fully enabled by the use of these relatively low amplitude and pressure shock waves. The method includes the steps of identifying high risk patients for a variety of potential infertility or impotence conditions. Such condition could be by way of example ovarian cancer treatments. After identifying a risk prone candidate providing one or a series of two or more exposure treatments with unfocused, divergent, planar or near planar shock waves or convergent far-sighted focused shock waves or diffused shock waves to the treatment site, in this example the region surrounding or in proximity to malignant cells or tumors. Then after treatments the physician can optionally ultrasound visually or otherwise determine the increase in regeneration or vascularization in the treated tissue after a period of time. Assuming an initial baseline determination of the tissue regeneration or vascularization had been initially conducted an estimate or calculation of treatment requirements can be made. If required, the physician can conduct a surgical procedure or alternatively prescribe medications. This procedure can be used for any at risk reproductive condition. After such a surgery or medical drug treatment the surrounding tissues can be post-operatively shock wave treated as well.

The implications of using the (re)generative features of this type of shock wave therapy are any weakened reproductive organ or tissue can be strengthened to the point of reducing or eliminating the risk of irreparable damage or degenerative failure.

The stimulation of growth factors and activation of healing acceleration within the cells of the treated tissues is particularly valuable to cancer patients and other high risk factor subjects exposed to radiation or chemical agents.

Even more striking as mentioned earlier, early prevention therapies can be employed to stimulate tissue or organ modeling to be maintained within acceptable ranges prior to an exposure to a degenerative condition occurring. This is extremely valuable in the prevention of age related infertility for example. The methods would be to identify at risk patients or workers based on family history and exposure risks, and subjecting that patient or worker to therapeutic shock wave therapy for the purpose of stimulating tissue repair or regeneration effectively remodeling the patient's susceptible organs to be within accepted functional parameters prior to exposure. The objective being to preventively stimulate cellular tissue repairs to pre-emptively avoid a degenerative condition from occurring which may result in the onset of cancer or other reproductive disease which may require invasive surgical procedures.

This preventive therapy is most needed to combat age related loss of function which left untreated results in cellular destruction or any other degenerative conditions within the reproductive system.

The following invention description first provides a detailed explanation of acoustic shock waves or pressure pulses, as illustrated in FIGS. 1-6. As used herein and shown in FIG. 6, an acoustic shock wave is an asymmetric wave with an exceptionally rapid peak rise time and slower return time from the peak amplitude. Historically, these acoustic shock waves or pressure pulses were first used medically to destroy kidney stones. The wave patterns were directed to a focal point at a relatively high energy to blast the concrements into small urinary tract passable fragments.

A whole class of acoustic shock waves or pressure pulses for medical treatments were later discovered that employed low energy acoustic shock waves or pressure pulses. These low energy acoustic shock waves or pressure pulses maintained the asymmetric wave profile, but at much lower energies.

As shown in FIG. 4, an exemplary view of an acoustic shock wave generator 1 is illustrated. The generator 1 has a control and power supply 41, a flexible hose 42 and an applicator 43.

These low energy acoustic shock waves or pressure pulses advantageously could stimulate a substance without requiring a focused beam. The advantage of such an unfocused beam was the acoustic wave could be directed to pass through tissue without causing any cell rupturing which would be evidenced by a lack of a hematoma or bruising. This use of unfocused, low energy acoustic shock waves or pressure pulses provided an ability to treat a large volume of tissue virtually painlessly. Furthermore, the acoustic energy caused a short duration anesthetic sensation that effectively numbs the patient's pain over a period of days with a prolonged reduction in pain thereafter.

The use of low energy acoustic shock waves or pressure pulses that employ a focused beam has been spurred on as a viable alternative to the unfocused low energy shock waves because the focal point being of a small zone of energy has little or a small region of cell damage as the remaining portions of the wave pattern can provide a stimulating effect similar to the unfocused shock waves. Basically, the effect is the same with the users of focused waves achieving the benefits of the unfocused waves, but with a focal point of peak energy in a tiny localised region. So, for purposes of the present invention, the use of “soft waves” those defined by low energy beams will be applicable to both focused and unfocused beams of acoustic shock waves or pressure pulses.

The asymmetric acoustic wave pattern shown in FIG. 7 is contrasted to an ultrasonic wave pattern which is illustrated in FIG. 6. As shown, ultrasound waves are symmetrical having the positive rise time equal to the negative in a sinusoidal wave form. These ultrasound waves generate heat in the tissue and are accordingly believed not suitable for use on harvested organs or tissue.

Heretofore such invasive techniques were not used in combination with shock wave therapy primarily because the shock waves were believed to be able to sufficiently pass through interfering body tissue to achieve the desired result in a non-invasive fashion. While this may be true, in many cases if the degenerative process is such that an operation is required then the combination of an operation in conjunction with shock wave therapy only enhances the therapeutic values and the healing process of the patient and the organ such that regenerative conditions can be achieved that would include not only revascularization of the heart or the reproductive organs wherein sufficient or insufficient blood flow is occurring but also to enhance the improvement of ischemic tissue that may be occupying a portion of the organ. This ischemic tissue can then be minimized by the regenerative process of using shock wave therapy in the fashion described above to permit the tissue to rebuild itself in the region that has been afflicted.

The use of these various acoustic shock wave forms can be used separately or in combination to achieve the desired therapeutic effect. Furthermore, such acoustic shock wave forms can be used in combination with drugs, chemical treatments, irradiation therapy or even physical therapy and when so combined the stimulated cells will more rapidly assist the body's natural healing response.

The present invention provides an apparatus for an effective treatment of indications, which benefit from low energy pressure pulse/shock waves having planar, nearly plane, convergent or even divergent characteristics. With an unfocused wave having planar, nearly plane wave characteristic, convergent or even divergent wave characteristics, the energy density of the wave may be or may be adjusted to be so low that side effects including pain are very minor or even do not exist at all.

In certain embodiments, the apparatus of the present invention is able to produce waves having energy density values that are below 0.1 mJ/mm² or even as low as 0.000 001 mJ/mm². In a preferred embodiment, those low end values range between 0.1-0.001 mJ/mm². With these low energy densities, side effects are reduced and the dose application is much more uniform. Additionally, the possibility of harming surface tissue is reduced when using an apparatus of the present invention that generates waves having planar, nearly plane, convergent or divergent characteristics and larger transmission areas compared to apparatuses using a target focused shock wave source that needs to be moved around to cover the affected target area. The apparatus of the present invention also may allow the user to make more precise energy density adjustments than an apparatus generating only focused shock waves, which is generally limited in terms of lowering the energy output.

With reference to FIGS. 1-3, a variety of schematic views of acoustic shock waves or pressure pulses are described. The following description of the proper amplitude and pressure pulse intensities of the shock waves 200 are provided below along with a description of how the shock waves actually function and have been taken from the co-pending application of the present inventors and replicated herein as described below. For the purpose of describing the shock waves 200 were used as exemplary and are intended to include all of the wave patterns discussed in the figures as possible treatment patterns.

FIG. 1 is a simplified depiction of a pressure pulse/shock wave (PP/SW) generator, such as a shock wave head, showing focusing characteristics of transmitted acoustic pressure pulses. Numeral 1 indicates the position of a generalized pressure pulse generator, which generates the pressure pulse and, via a focusing element, focuses it outside the housing to treat diseases. The affected infected tissue or organ is generally located in or near the focal point which is located in or near position 6. At position 17 a water cushion or any other kind of exit window for the acoustical energy is located.

FIG. 2 is a simplified depiction of a pressure pulse/shock wave generator, such as a shock wave head, with plane wave characteristics. Numeral 1 indicates the position of a pressure pulse generator according to the present invention, which generates a pressure pulse which is leaving the housing at the position 17, which may be a water cushion or any other kind of exit window. Somewhat even (also referred to herein as “disturbed”) wave characteristics can be generated, in case a paraboloid is used as a reflecting element, with a zone source (e.g. electrode) that is located in the focal point of the paraboloid. The waves 200 can be transmitted into harvested organs or tissue via a coupling media such as, e.g., ultrasound gel or oil and their amplitudes will be attenuated with increasing distance from the exit window 17.

FIG. 3 is a simplified depiction of a pressure pulse shock wave generator (shock wave head) with divergent wave characteristics. The divergent wave fronts may be leaving the exit window 17 at zone 11 where the amplitude of the wave front is very high. This zone 17 could be regarded as the source zone for the pressure pulses.

This apparatus, in certain embodiments, may be adjusted/modified/or the complete shock wave head or part of it may be exchanged so that the desired and/or optimal acoustic profile such as one having wave fronts with focused, planar, nearly plane, convergent or divergent characteristics can be chosen.

A change of the wave front characteristics may, for example, be achieved by changing the distance of the exit acoustic window relative to the reflector, by changing the reflector geometry, by introducing certain lenses or by removing elements such as lenses that modify the waves produced by a pressure pulse/shock wave generating element. Exemplary pressure pulse/shock wave sources that can, for example, be exchanged for each other to allow an apparatus to generate waves having different wave front characteristics are described in detail below.

In one embodiment, mechanical elements that are exchanged to achieve a change in wave front characteristics include the primary pressure pulse generating element, the focusing element, the reflecting element, the housing and the membrane. In another embodiment, the mechanical elements further include a closed fluid volume within the housing in which the pressure pulse is formed and transmitted through the exit window.

In one embodiment, the apparatus of the present invention is used in combination therapy. Here, the characteristics of waves emitted by the apparatus are switched from, for example, focused to divergent or from divergent with lower energy density to divergent with higher energy density. Thus, effects of a pressure pulse treatment can be optimized by using waves having different characteristics and/or energy densities, respectively.

While the above described universal toolbox of the various types of acoustic shock waves or pressure pulses and types of shock wave generating heads provides versatility, the person skilled in the art will appreciate that apparatuses that produce acoustic shock waves or pressure pulses having, for one example, nearly plane characteristics, are less mechanically demanding and fulfil the requirements of many users.

As the person skilled in the art will also appreciate that embodiments shown in the drawings are independent of the generation principle and thus are valid for not only electro-hydraulic shock wave generation but also for, but not limited to, PP/SW generation based on electromagnetic, piezoceramic and ballistic principles. The pressure pulse generators may, in certain embodiments, be equipped with a water cushion that houses water which defines the path of pressure pulse waves that is, through which those waves are transmitted. In a preferred embodiment, the organ or tissue directly or the fluid filled container holding these harvested organs or tissue is coupled via ultrasound gel or oil to the acoustic exit window (17), which can, for example, be an acoustic transparent membrane, a water cushion, a plastic plate or a metal plate.

In the pressure pulse or shock wave method of treating an infection within a harvested tissue or organ with a risk of exposure to an infection or post-occurrence of such infections requires the harvested tissue or organ to be positioned in a convenient orientation to permit the source of the emitted waves to most directly send the waves to the target site to initiate pressure pulse or shock wave stimulation of the target area with minimal, preferably no obstructing features in the path of the emitting source or lens other than the fluid filled container holding the organ or tissue. Assuming the infection target area or site is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent, planar or near planar and having a low pressure amplitude and density in the range of 0.00001 mJ/mm² to 1.0 mJ/mm² or less, most typically below 0.2 mJ/mm². The focused source preferably can use a diffusing lens or have a far-sight focus to minimize if not eliminate having the localized focus point within the tissue. Preferably the focused shock waves are used at a similarly effective low energy transmission or alternatively can be at higher energy but wherein the tissue target site is disposed pre-convergence inward of the geometric focal point of the emitted wave transmission. In treating some hard to penetrate infections, the pressure pulse more preferably is a high energy target focused wave pattern which can effectively attack the infection outer structure or barrier shield causing fractures or openings to be created to expose the colonies of microorganisms within the infection to the germicidal effects of the pressure pulses or shock waves. This emitted energy destroys the underlying microorganism's cellular membranes. In addition, the fragmentation of the infections outer barrier is then easily germicidally killed out of the harvested material. The surrounding healthy cells in the region treated are activated initiating a defense mechanism response to assist in eradication of any unwanted infection.

These shock wave energy transmissions are effective in stimulating a cellular response and can be accomplished without creating the cavitation bubbles in the tissue of the target site when employed in other than high energy focused transmissions. This effectively ensures the tissue or organ does not have to experience the sensation of hemorrhaging so common in the higher energy focused wave forms having a focal point at or within the targeted treatment site.

The limiting factor in the selected treatment dosage is to provide a stimulating stem cell activation or a cellular release or activation of the LL37 protein and VEGF and other growth factors while simultaneously germicidally attacking the infection barrier and any underlying colony of microorganisms.

FIG. 4 shows an exemplary shock wave device generator or source 1 with a control and power supply 41 connected to a hand-held applicator shock wave head 43 via a flexible hose 42 with fluid conduits. The illustrated shock wave applicator 43 has a flexible membrane at an end of the applicator 43 which transmits the acoustic waves when coupled to the external surface of the harvested organ or tissue or the fluid filled container holding this harvested material by using a fluid or acoustic gel. As shown, this type of applicator 43 has a hydraulic spark generator using either focused or unfocused shock waves, preferably in a low energy level, less than the range of 0.01 mJ/mm² to 0.3 mJ/mm². The flexible hose 42 is connected to a fluid supply that fills the applicator 43 and expands the flexible membrane when filled. Alternatively, a ballistic, piezoelectric or spherical acoustic shock wave device can be used to generate the desired waves.

In the figures, exemplary shock waves 200 are illustrated, it must be appreciated that any of the recognized shock wave patterns can be used in the shock wave treatment of the various penile organs or tissues 100.

As illustrated, the device 1 shown is an electrohydraulic acoustic shock wave generator 1, however, other devices that generate acoustic shock waves or pressure pulses can be used. Ultrasonic devices may be considered, but there is no data to support a sinusoidal wave form would work and therefore not considered as effective as the asymmetric wave generators. The acoustic shock waves or pressure pulses activate a cellular response within the reflexology treatment site. This response or stimulation causes an increase of nitric oxide and a release of a variety of growth factors such as VEGF and a release of anti-microbial peptides like LL37. As shown, the flexible membrane is protruding outward and the applicator 43 has been filled with fluid, the transmission or emission of acoustic shock waves or pressure pulses 200 is directed towards the penis 100.

The transmission of the shock waves 200 can be of a low energy density of 0.2 mJ/mm² whether using focused or unfocused shock waves. The acoustic shock waves or pressure pulses pulse rapidly through the cells penetrating the cell membrane extremely rapidly due to the rapid rise to peak time and pass through exiting slower due to the slower return from peak amplitude. This asymmetric wave pattern rapidly compresses each cell on entry and slow decompresses the cell as it exits. This effective squeezing of each cell is believed to cause the release of growth factors such as VEGF and others and also creates nitric oxide, all beneficial to new blood vessel formation. This occurs as a transmission across the cell membranes without rupturing the native cells.

Furthermore, such acoustic shock wave forms can be used in combination with drugs, chemical treatments, irradiation therapy or even physical therapy and when so combined the stimulated cells will more rapidly assist the body's natural healing response and thus overcomes the otherwise potentially tissue damaging effects of these complimentary procedures.

The present invention provides an apparatus for an effective treatment of indications in harvested organs or tissues, which benefit from high or low energy pressure pulse/shock waves having focused or unfocused, nearly plane, convergent or even divergent characteristics. With an unfocused wave having nearly plane, plane, convergent wave characteristic or even divergent wave characteristics, the energy density of the wave may be or may be adjusted to be so low that side effects including pain are very minor or even do not exist at all.

In certain embodiments, the apparatus of the present invention is able to produce waves having energy density values that are below 0.1 mJ/mm² or even as low as 0.000001 mJ/mm². In a preferred embodiment, those low end values range between 0.1-0.001 mJ/mm². With these low energy densities, side effects are reduced and the dose application is much more uniform. Additionally, the possibility of harming surface tissue is reduced when using an apparatus of the present invention that generates unfocused waves having planar, nearly plane, convergent or divergent characteristics and larger transmission areas compared to apparatuses using a focused shock wave source that need to be moved around to cover the affected area. The apparatus of the present invention also may allow the user to make more precise energy density adjustments than an apparatus generating only focused shock waves, which is generally limited in terms of lowering the energy output. Nevertheless, in some cases the first use of a high energy focused shock wave targeting a treatment zone may be the best approach followed by a transmission of lower energy unfocused wave patterns.

The penile tissue is strengthened, inflammation reduced, nerves regenerated, and stem cells recruited and activated. All acoustic waves, focused and unfocused, spherical, radial, ballistic, etc. could be used for treatments.

It will be appreciated that the apparatuses and processes of the present invention can have a variety of embodiments, only a few of which are disclosed herein. It will be apparent to the artisan that other embodiments exist and do not depart from the spirit of the invention. Thus, the described embodiments are illustrative and should not be construed as restrictive.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of treatment for a penis of an adult postpubertal male patient, the treatment comprising of the steps of:
causing an erection of a penis;
activating an acoustic shock wave generator or source to emit low energy or unfocused acoustic shock waves, wherein the acoustic shock waves are waves having amplitudes above 0.1 MPa and rise times of the amplitude are below 100 nano-seconds with a duration of a shock wave being below 3 micro-seconds for the positive part of a cycle and wherein the pressure pulses are an acoustic pulse which includes several cycles of positive and negative pressure with amplitudes of the positive part of such a cycle being above 0.1 MPa and the pressure pulse time duration is from below a microsecond to about a second, rise times of the positive part of the first pressure cycle is in the range of nano-seconds up to several milli-seconds; and
subjecting the erect penis to the acoustic shock waves stimulating said erect penis, the erect penis is positioned within a path of the emitted shock waves stimulating a cellular response.

2. The method of treatment of claim 1 wherein the emitted shock waves or pressure pulses are convergent, divergent, planar or near planar.

3. The method of treatment of claim 1 wherein the emitted shock waves or pressure pulses are convergent having one or more geometric focal volumes or points located at a distance X, X being defined as the distance from an exit window to the one or more focal volumes or points from the generator or source, the erect penis being positioned at the distance X or less than the distance X from the exit window source.

4. A method of treatment for increasing testosterone levels and increasing sperm count and viability in an adult postpubertal male patient comprising of the steps of:
testing the testosterone and sperm count and viability of a patient prior to treatment to establish a pre-treatment baseline level;
activating an acoustic shock wave or pressure pulse generator or source to emit low energy or unfocused acoustic shock waves or pressure pulses, wherein the acoustic shock waves are waves having amplitudes above 0.1 MPa and rise times of the amplitude are below 100 nano-seconds with a duration of a shock wave being below 3 micro-seconds for the positive part of a cycle and wherein the pressure pulses are an acoustic pulse which includes several cycles of positive and negative pressure with amplitudes of the positive part of such a cycle being above 0.1 MPa and the pressure pulse time duration is from below a microsecond to about a second, rise times of the positive part of the first pressure cycle is in the range of nano-seconds up to several milli-seconds;
subjecting a testicle through the scrotum to the acoustic shock waves or pressure pulses stimulating said testicle to increase testosterone levels and increase sperm count and viability, wherein the testicle is positioned within a path of the emitted shock waves stimulating a cellular response without creating cavitation bubbles insuring no hemorrhaging;
testing the testosterone level and sperm count and viability of the patient within 72 hours after subjecting the testicle to the acoustic shock waves or pressure pulses to establish a post treatment level; and
comparing the pre-treatment baseline level to the post treatment level tested after treatment to establish any increase.
